SEQUENCE LISTING (3 sequences)

SEQ ID NO: 1 - DNA, 29 bp, Artificial Sequence
  Primer
tcgtggtggacttctctcaattttctagg

SEQ ID NO: 2 - DNA, 26 bp, Artificial Sequence
  Primer
cgaggcatagcagcaggatgaagaga

SEQ ID NO: 3 - DNA, 2861 bp, Artificial Sequence
  Synthetic construct
aattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcctgcaggtctcgtggtggacttctctcaattttctagggggaacacccgtgtgtcttggccaaaattcgcagtcccaaatctccagtcactcaccaacttgttgtcctccgatttgtcctggttatcgctggatgtgtctgcggcgttttatcatctttctcttcatcctgctgctatgcctcggactctagaggatccccgggtaccgagctcg

(19) United States
(12) Patent Application Publication
(10) Pub. No.: US 2009/0118484 A1
(43) Pub. Date: May 7, 2009

(54) FORMATION OF NOVEL NUCLEIC ACID COMPLEXES AND DETECTION THEREOF

(76) Inventor: Chang-ning J. Wang, Andover, MA (US)

Correspondence Address: Kristina M. Grasso;Kristina M. Grasso, Esq. PLLC, P.O. Box 162, Milford, NH, 03055, US

(21) Appl. No.: 11/970,001

(22) Filed: Jan. 7, 2008

Related U.S. Application Data
(63) Continuation-in-part of application No. 11/069,370, filed on Feb. 28, 2005, now abandoned.
(60) Provisional application No. 60/548,963, filed on Feb. 28, 2004.

(51) Int. Cl. C07H 21/00

(52) U.S. Cl. 536/23.1

(57) ABSTRACT

This invention relates to a process and system to amplify and detect recombinational non-reciprocal cross-over reactions between homologous nucleic acid molecules without the assistance from a protein factor. The result of a chain reaction of non-reciprocal cross-overs is stoichiometrical formation of nucleic acid conglomerate complex that binds significantly more ethidium bromide or other fluorophores than a canonical B-form double helical nucleic acid does, emitting much stronger fluorescence. Such nucleic acid conglomerate complex can be easily detected by conventional methods, therefore can be used to detect any target molecule of interest.

### RELATED APPLICATION

This application claims priority to co-pending U.S. Nonprovisional Patent Application Ser. No. 11/069,370 filed on Feb. 28, 2005, which claims priority to U.S. Provisional Application Ser. No. 60/548,963, filed on Feb. 28, 2004. The content of these applications are incorporated by reference in their entireties.

### TECHNOLOGY FIELD

This disclosure relates to nucleic acid manipulations, more specifically, it relates to a process and system to amplify and detect recombinational non-reciprocal cross-over reactions between conformationally different homologous nucleic acid molecules without the assistance of a protein factor. The result of such chain reaction of non-reciprocal cross-overs is the stoichiometrical formation of a nucleic acid conglomerate complex that binds significantly more ethidium bromide or other fluoroscopes than the canonical B-form double helical nucleic acid does, thus emitting a much stronger fluorescence. Such nucleic acid conglomerate complex can be easily detected by conventional methods, thereby greatly increasing the sensitivity in detecting a homologous nucleic acid molecule.

### BACKGROUND

A particular nucleic acid molecule may contain information about cancer, infectious disease, gene structure, genetic disease, or hair color etc. Manipulation of nucleic acid is a fundamental component of modem molecular biotechnology, either for understanding the nature of life or for controlling and monitoring diseases. Over the years, many techniques have been developed for detecting a particular nucleic acid molecule in a given specimen.

Among them, probe hybridization, where a target nucleic acid molecule may be detected by binding to its complementary probe nucleic acid molecules, has been used for detecting the existence of very small amounts of a particular nucleic acid molecule. This technique, as the classic technique, has been used by many researchers, scientists and clinicians. However, this method is in fact not very sensitive; often fails to distinguish true signals from noisy non-specific binding. This method is also time consuming, requires 12-16 hours of hybridization and needs temperature controlled hybridization oven and often radioactively labeled probes.

There are a few methods that can improve the sensitivity of hybridization detection. Methods such as further amplifying signals, cycling targets, cycling probes, or using branched DNA molecules as a signal generator. See, e.g., U.S. Pat. Nos. 4,699,876, 6,114,117, and 5,118,605; and Bekkaoui, et al., Bio-Techniques, 20: 240-248, 1996. However, owing to the fact that these techniques would also indiscriminately amplify background noises intrinsic to nucleic acid hybridization, these methods may not be satisfactory in certain applications.

An alternative widely used method is Polymerase Chain Reaction (PCR) which improves detection sensitivity by repeated de novo synthesis of a specific target sequence. See, e.g., U.S. Pat. Nos. 4,683,195, 4,683,202, 4,800,159, 5,455,166, 5,288,611, 5,639,604, 5,658,737, and 5,854,033; EP No. 320,308 A2; International Application PCT/US87/00880, and Zehbe et al., 20 Cell Vision, vol. 1, No. 1, 1994. However, a PCR reaction requires expensive instruments and enzymes, and can also be time consuming. Depending on the batch and type of polymerase enzymes the result of the detection may be not reliable.

Recombinase recA-based homologous DNA strand exchange and D-loop formation have also been utilized to enrich and detect nucleic acids. See, e.g., U.S. Pat. Nos. 5,670,316 and 6,335,164. These methods, however, are susceptible to interference by heterozygous DNA.

Moreover, the success of above methods hinges on the integrity of the target sequence. The stringent quality requirements of the target molecule severely limit their application to only good, well-preserved samples. Damaged samples will generate unreliable results or no results.

Therefore, there is a need to develop a novel nucleic acid detection method that is more sensitive, specific, and inexpensive.

This disclosure identifies a novel process to form a nucleic acid complex and the detection of such complex formation provides a sensitive, inexpensive way to detect a target molecule of interest.

### SUMMARY

The present invention is based on applicant's discovery that under a paranemic denaturing condition at the interfacial surface between an aqueous phase and a hydrophobic phase, a nucleic acid molecule of different conformation mixed with a mass of homologous double-stranded molecules triggers a chain reaction of homologous recombinational non-reciprocal crossovers and strand displacements. This reaction results in forming a stable, compact conglomerate nucleic acid complex or particle. The nucleic acid strands in the complex still engage in Watson-Crick pairing, but may adopt non-canonical double helical structure, which, when stained with a fluorescent dye binds much more fluoroscope molecules producing highly enhanced fluorescence emission. Such a particle is readily observable under a light microscope with distinct shapes, and can be easily spun down by a bench top microcentrifuge.

The formation of this nucleic acid particle can thus be used to easily detect a target nucleic acid molecule in a sample of interest.

Further, because a single molecule is sufficient to trigger the chain reaction, the formation of such nucleic acid complex can be observed with the existence of even one target molecule. This method of detection is so sensitive that it will detect one single target molecule. Because fragmented target sequences also trigger the chain reaction, target molecule sequence integrity would not significantly affect the efficiency of this method of detection.

In one embodiment, the process for forming a conglomerate nucleic acid complex particle is by mixing in a paramedic denaturing chaotropic aqueous solution, preferably a molar excess of homologous probe nucleic acid molecules with a sample that contains a target nucleic acid molecule. Each probe molecule should have a complementary strand and a strand having a sequence homologous to the target sequence. A sufficient homology between the probe molecule and the target molecule is preferably greater than 18 base pairs.

The sufficient ratio of the reactants for the probe and target molecules is dictated by the final stoichiometry for the components of the conglomerate nucleic acid complex. In general, molar excess of probe molecules is sufficient to ensure successful conversion of individual conformers into the conglomerate nucleic acid complex for most target sequences.

When a chaotropic solution containing nucleic acid molecules is admixed with a hydrophobic solvent, nucleic acid strands are adsorbed onto the interfacial surface between the hydrophobic phase and the chaotropic aqueous solution. The hydrophobic bases of the nucleic acid molecules are intended to bury into the hydrophobic solvent, while the hydrophilic phosphoribose backbones are dragged towards the adjacent aqueous phase. The adsorbed nucleic acid molecules are thus prostrated by the surface tension, forcing each molecule to lie side by side. Such spatial arrangement will accelerate the Watson-Crick base pairing process to form paranemic structure between complementary strands of nucleic acids by drastically limiting plausible conformations of nucleic acid molecules to a subset of transient intermediates and establishing a homeostasis of dynamic interactions between multiple polemic double stranded pairs. Such structural arrangement would lower the energy barrier for strand invasion and displacement, making it much easier for homologous recombination.

In a homogenous population of paranemic denatured probe molecules, each strand of the probe molecule is in the paranemic homeostasis of interaction with its adjacent molecules. That is, most of the strand invasion and displacement reactions are reciprocal and reversible. And for a period of time, this conformational homeostasis between the polemically denatured probe molecules will maintain stability. However, in the presence of a homologous target molecule that has different conformation to the homologous probe molecules, the pairing of the target molecule with the complementary strand of a probe molecule will induce local conformational perturbation of the probe molecule. The conformational discontinuity formed at the junction of the target-probe hybrid will delay the probe molecule conformation dynamics, and allow sufficient time for the distortion torque generated at the junction of the phosphoribose backbone to cause the hybrid molecule to enter the aqueous phase. And in the aqueous phase the paired hybrid strands will freely intertwine and form a stable double helix. The displaced strand of the invaded probe molecule still at the interfacial surface will now search for a stable conformation under the environment. If the newly assumed conformation of the displaced probe strand is different that that adopted by its surrounding paranemic homologous molecules, the next round of productive strand invasion and strand displacement will ensue. Such a reaction cycle will continue until the paranemic probes at the interface are depleted as a substrate for further strand invasion or the interfacial adsorption force is no longer strong enough to hold the newly formed conglomerate nucleic acid complex at the interfacial surface.

In one embodiment, a different conformation between a probe molecule and a target molecule is present because they were from different sources.

In another embodiment, there is a different conformation between a probe molecule and a target molecule because they were exposed to different chemical or physical treatments.

In one embodiment, the premix denaturization is achieved by mixing chaotropic aqueous solution containing both probe molecules and target molecules with a hydrophobic solvent such as aniline or phenol.

The types of probe molecules and target molecules may include DNA, RNA, PNA, hybrids, or derivatives thereof that are capable of forming Watson-Crick bonds and nucleotide pairing. The target molecule can either be single stranded, double stranded or multiple stranded, while probe molecules should have at least two complementary strands. The probe molecule may also be in a circular form without free ends.

In another embodiment, the formation of the nucleic acid complex particles is proportional to the copy number of a target molecule in the excess of probe molecules. This close linear relationship may be used to quantitatively detect the presence of a specific target molecule.

In another embodiment, the target molecule is fragmented.

In another embodiment, the target molecules are nucleic acids from tissue samples or body fluids, such as blood, urine, saliva, lymphoid fluid, sweat etc. The target molecule may include, but is not limited to, sequences such as sequences of cancer genes, genes of cell cycle, genes of hormones, genes of membrane proteins, genes encoding signal transduction pathways, sequences of regulatory functions, genes related to metabolic diseases, infectious viruses, genes that are gender specific, genes related to Down's syndrome, genetic diseases, sequences that are specific for identity markers and other sequences that are detectable by hybridization or PCR.

In another embodiment, the mixture of denatured nucleic acid may have several different species of probes having sequences homologous to several different target sequences; these probes are labeled with different fluorescent dyes.

In another embodiment, the formation of the conglomerate nucleic acid crossover complexes are detected by staining with a fluorophore compound, such as ethidium bromide or PicoGreen, and/or may be detected by light microscopy, fluorescence microscopy or gel electrophoresis.

In another embodiment, the formation of the conglomerate nucleic acid crossover complexes are detected by direct centrifuging alone without other manipulation.

In another embodiment, the formation of the conglomerate nucleic acid crossover complexes are detected by quantitative PCR.

Other features, objects, and advantages of the invention will be apparent from the description and from the claims.

### DESCRIPTION OF THE DRAWINGS

FIG. 1 is a schematic diagram of the initial several rounds of nucleic acid homologous recombination cross-overs occurring at the interfacial surface between a chaotropic aqueous solution and a hydrophobic solvent.

FIG. 2 shows microscopic photographs of the formed conglomerate nucleic acid recombination cross-over complexes of Example 1. FIG. 2a is a photograph taken under an optical microscope. FIG. 2b is a photograph taken under a fluorescence microscope.

FIG. 3. is a photograph of an ethidium bromide stained agarose gel displaying the electrophoresis pattern of the homologous recombination cross-over complex of Example 1 in comparison to double stranded and single stranded probe nucleic acid molecules.

FIG. 4 is the measurement of the stoichiometrical relationship between the copy number of the target molecules and the copy number of the probe molecules in Example 1 by quantitative polymerase chain reaction

### DETAILED DESCRIPTION

1. Definitions

The term “interfacial surface” refers to the two-dimensional surface between two immiscible phases, such as between all aqueous phase and a hydrophobic phase.

The term “nucleic acid” refers to ribonucleic acid (RNA), deoxyribonucleic acid (DNA) and their hybrids and derivatives, such as peptide nucleic acid (PNA), labeled and modified nucleic acid molecules.

The term “paranemically denatured” or “paranemically denaturing” refers to the conditions in which nucleic acid participating strands lay side by side and can be separated without mutual rotation of the opposite strands, while complementary strands remain Watson-Crick base pairing. Nucleic acid duplex strands are paranemically denatured at the interfacial surface between an aqueous phase and a hydrophobic phase. Experimental detecting methods includes probing by single-strand specific nucleates (SNN), conformation-specific chemical probes, topoisomer analysis, NMR, and other physical methods. Paranemic structure has been found in single-stranded DNA, slippage structures, cruciforms, alternating B-Z regions, triplexes (HDNA), paranemic duplexes, RNA, and protein-stabilized paranemic DNA (Yagli G. “Paranemic structures of DNA and their role in DNA unwinding”, Crit Rev Biochem Mol Biol. 1991 Vol 26, No 5-6, pages 475-559).

The term “conformation” refers to the structures other than the primary sequence composition and order. Conformation of a nucleic acid strand may be different because of any prior different environments, such as different origins, different extractions, different batches of preparations, different temperatures, different magnetic fields, different light waves, different ion strengths, different organic or inorganic solvent exposures, different interactions with other molecules, etc.

The term “reciprocal cross-over” refers to the crossover between two nucleic acid molecules (duplex or multistrand) wherein strand(s) from each molecule invade each other to form a crossover strand exchange.

The term “non-reciprocal cross-over” refers to the crossover between two nucleic acid molecules (duplex or multistrand) wherein strand(s) of one of the molecules invades the other molecule, and the strands of the other molecule do not invade back.

The term “conglomerate crossover complex” refers to a multistrand containing nucleic acid complex (more than two pairs of duplex nucleic acid molecules or multistrand molecules) connected together by plurality of non-reciprocal cross-overs. Strands of different molecules are then joined to form a conglomerate nucleic acid complex.

The term “synapsis clamp locus” refers to a stretch of nucleic acid sequence that features a distinct energy barrier capable of stalling the cross-over junctions from branch migration.

2. Introduction

E. Coli RecA protein initiates homologous recombination by paring homologous DNA molecules to form polemic joints (Wong et al, “The role of negative superhelicity and length of homology in the formation of paranemic joints promoted by RecA protein”, J. Biol. Chem. 1998 May, Vol 273, No 20, pages 12120-27). Other recombinases and mediators, such as Rad 51, facilitate homologous recombination by stabilizing single strand nucleic molecule to invade a homologous duplex during the formation of a D-loop structure (see Sung P. et al., Rad 51 Recombinase and Recombination Mediators, J. Biol. Chem., 2003, Vol. 278, No. 44, pp 42729-32).

Other references disclose conglomerate nucleic acid particles comprising nucleic acid probe molecules, wherein the probe molecules have at least two complementary strands. See e.g. U.S. Pat. No. 6,255,469. However, these particles are very small, producing a complex with four strands and free ends, with no indication of enhancement of its fluorescence after staining. This type of structure cannot be observed or distinguished under a light microscope. Additionally, such a structure cannot propagate and will always end up with a duplex structure with an even number of strands. In the present invention, the process initiated with a single strand target will generate an odd number of strands, and will allow for continuous propagation. For example, if P=probe and T=target, then the structure described in U.S. Pat. No. 6,255,469 would be 2P+2T or 2(P+T). In the present invention, the structure would be comprised of 2P+1T.

However, homologous recombinational chain reaction without a protein factor has never been reported. This disclosure identifies a novel system and method that produces stable paranemic duplex nucleic acid structures without a protein factor by conducting interfacial homologous recombination chain reactions. Because these interfacial homologous recombinational chain reactions do not occur without a triggering homologous molecule from a different source, this method and system can be used to recognize and detect homologous molecules that come from a different source. Additionally, because the difference between the trigger molecule and the corresponding probe molecules may only be attributed structurally to their different conformations, this system and method can be reasonably used to recognize and detect any homologous molecules that are conformationally different from the probe molecules.

3. General Mechanism

The following describes generally how the method and system works.

A target site or molecule can be a part of a single stranded nucleic acid (e.g., a single stranded viral DNA or a human RNA), a double stranded DNA (e.g., a double stranded viral DNA or a human genomic DNA), a DNA-RNA hybrid, and combinations and derivatives of one or more of the above. For example, a double stranded viral DNA isolated from human blood can be detected as follows

One first selects a target site to be detected by identifying a consensus sequence of the viral genome that is unique to the virus and absent from human and other viral DNAs. One then constructs by standard molecular cloning techniques a double stranded DNA probe containing a sequence complementary to the selected target site. The double stranded viral DNA and the double stranded probe (the amount of probe may be determined empirically, preferably, in molar excess) are then denatured or partially denatured in a chaotropic aqueous solvent, in which the viral DNA and the probe are destabilized and their respective complementary strands dissociate to adopt an unwound conformation. A hydrophobic solvent is then added to the aqueous solvent to create a biphasic system in which a planar surface is formed between the two solvents. Examples of suitable hydrophobic solvents include aniline, n-butylalcohol, tert-amylalcohol, cyclohexyl alcohol, phenol, p-methoxyphenol, benzyl alcohol, pyridine, purine, 3-aminotriazole, butyramide, hexamide, thioacetamide, 8-valarolactum, tert-butylurea, ethylenethiourea; allylthiourea, thiourea, urethane, silicones, N-propylurethane, N-methylurethane, cyanoguanidine, and combinations of two or more of the above. Examples of suitable chaotropic aqueous solvents include those containing one or more of SCN⁻, Mg²⁺, Ca²⁺, Na⁺, K⁺, NH₄, Cs⁺, Li⁺, and (CH₃)₄N⁺, Guanidinium ion in combination with those containing one or more of tosylate, —ClCCOO⁻ ClO₄, Br⁻ Cl⁻ BrO₃⁻, CH₃COO⁻, HSO₃⁻, F⁻, SO₄²⁻, (CH₃)₃CCOO⁻, and HPO₄⁻.

The double stranded probe and the double stranded viral DNA in a chaotropic aqueous solvent-hydrophobic organic solvent system (i.e., an amphipathic environment) will be attracted to the interfacial surface between the organic and aqueous phases and expose their hydrophilic phosphoribose moieties to the aqueous phase and their hydrophobic ring moieties to the hydrophobic phase. The interfacial tension therefore will keep all nucleic acid molecules on the same planar surface in a side-by side spatial arrangement. The complementary strands of homogenous probe molecules will therefore maintain a stable homeostasis of dynamic interactions in paranemic pairing. With the presence of a target site-containing viral DNA of a different source or conformation, regional pairing balance between a pair of paranemic probe strands will be disrupted, initiating a series of strand displacement reaction.

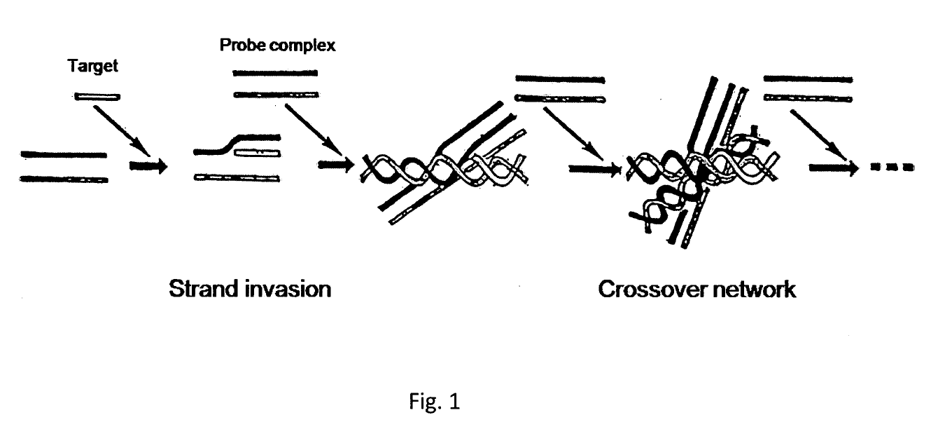

As shown in FIG. 1, the invasion of the target viral DNA strand 10 by Watson-Crick pairing with the complementary probe strand 20, partially displaces region 31 of corresponding probe strand 30, forming a non-reciprocal crossover at the junction. The formed crossover at junction distorts the topological structure of the paranemic probe strands and forces parts of them from the planar surface into the aqueous phase. Upon entering into the aqueous phase, the paired strands will freely intertwine, forming a more stable helical duplex 60, which releases free energy to allow the displaced single strand part 31 of the probe molecule to invade another probe molecule 70 which contains paranemically paired strands 40 and 50 on the interfacial surface. This invasion forms another non-reciprocal crossover at the junction, and thus another stable helical duplex 80 in the aqueous phase. This cycle will continue until the paranemic probe molecules are depleted or the formed conglomerate crossover complex is too big to stay at the interfacial surface. With molar excess of probe molecules, the reaction is stoichiometrical between the probe molecules and target molecules.

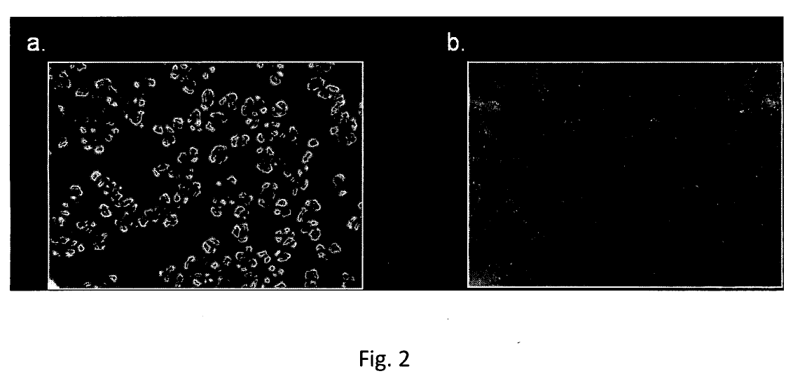

The complex thus formed can be easily isolated by ethanol or isopropanol precipitation in the presence of a chaotropic aqueous solution, or by direct centrifuging. The formed complex can also be observed under light microscope and fluorescent microscope as shown in FIG. 2a and FIG. 2b. Because one molecule is sufficient to trigger the described chain reaction, the system can be easily scaled to the sensitivity of testing the existence of a single target molecule of interest in a sample.

The complex thus obtained, when excited at 518 nm after staining with ethidium bromide, emits fluorescence at 605 nm with an intensity at least 10 times that of ethidium bromide-stained conical B-form nucleic acid helixes. In essence, this much enhanced fluorescence intensity can be determined as follows: 100 ng of the conglomerate crossover complex is stained with 0.25 μg/ml ethidium bromide for 5 minutes. The fluorescence intensity is then measured and compared with that obtained from 100 ng of the non-processed nucleic acids, i.e., the viral DNA and the double stranded probe (data not shown)

The complex can also be detected by other methods. For example, it can be visualized after resolving by gel electrophoresis. Presence of the relaxed crossover complex is indicated by a band on the gel with a molecular weight larger than the combined molecular weights of the non-processed nucleic acids.

Alternatively, the complex can be detected as a species farther removed from the axis of rotation as compared to the non-processed nucleic acids during sedimentation equilibrium process.

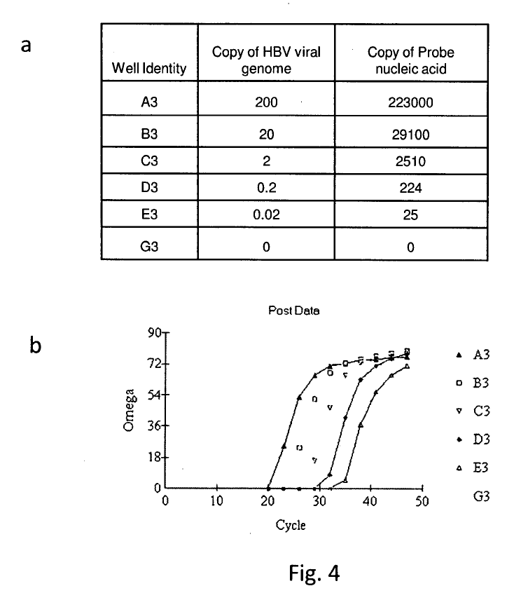

One can also detect the complex by microscopy. For example, fluorescent dye stained complex can be observed under a fluorescence microscope after moisture chamber vaporization on a microscope slide. The amount of complex formed can also be quantified by quantitative PCR (QPCR) after the on-reacted probe, present in single stranded form, was removed by digestion with a single stranded DNA specific nucleate (e.g., Mung bean nuclease). Probe-specific primers can be used to amplify the target sequences in the presence of signal generating primer (e.g. Amplisensor) or probe (e.g. TaqMan probe) to reveal the total amount of the probe nucleic acid engaged in the complex (see FIG. 4)

Nucleic acids from biological or other samples do not necessarily have to be purified prior to the detection assay. For example, a sample (e.g., blood, lymphatic fluid, urine, food, or sewage) may be first incubated in a lysis buffer, and then be directly ethanol or isopropanol precipitated. The supernatant of resuspension may be directly used for detection. Additionally, an unprocessed sample may be directly used for detection. The concentration of the chaotropic agent(s) can be determined empirically such that the complementary strands of the probe nucleic acids, after the denaturation, still pair with each other side by side on a planar surface.

In general, detection sensitivity can be improved by augmenting the quantity or increasing the length the probe strands. By including a high energy barrier in the probe, i.e. a synapsis clamp locus region to prevent branch migration, it will also stabilize the complex after it has been formed. One can also increase detection sensitivity by repeating the complex formation process after fragmenting the complex prior to each repeat of the process. A conglomerate complex can be fragmented with T7 endonuclease I, which digests mismatched DNA and some of the crossover structures. A fragmented complex will resume a canonical B form helix, can thus be made paranemic at the interfacial surface and trigger the homologous recombination crossover reactions with a new supply of double stranded probes.

One can use multiple types of probes specific to a single target site or a single type of probe specific to multiple target sites to detect different target sites in one single assay. Since the signal will be directly related to the amount of individual target sites present in the reaction, this approach is particularly useful for simultaneously detecting multiple pathogens in a sample.

The method and system described above can also be used to prepare coating material by replacing, if necessary, the viral DNA with any suitable nucleic acid sequences, The conglomerate crossover complex has a high charge density due to the polyanionic groups of the nucleic acids, and can be used to coat a surface for immobilizing cationic molecules. It can be applied to a surface as a thin-film by standard spraying techniques.

Also within the scope of this invention is a kit for detecting specific nucleic acid sequences by the above-described process. The kit can contain two or more of the following reagents: a probe nucleic acid specific to a target sequence, a chaotropic reagent or a chaotropic aqueous solvent, a hydrophobic solvent, and a fluorescent dye for detection.

Without further elaboration, it is believed that one skilled in the art can, based on the description above (including a postulated mechanism, which does not restrict the scope of this invention as claimed), utilize the present invention to its fullest extent. All publications cited herein are hereby incorporated by reference in their entirety. The specific example below is to be construed as merely illustrative, and not limitative of the remainder of the disclosure in any way whatsoever.

### EXAMPLE 1

Human hepatitis B virus (HBV) genome was detected using a nucleic acid sequence that contained a segment as probe corresponding to the HBV surface antigen specific sequence (HBVSAg). This HBVSAg segment was PCR-amplified from a source of the HBV genome using the following primers: TCG TGG TGG ACT TCT CTC AAT TTT CTAGG, (SEQ ID NO: 1) and CGA GGC ATA GCA GCA GGA TGA AGA GA (SEQ ID NO. 2). The PCR-amplified HBVSAg segment was then sub-cloned into a modified PUC18 plasmid via a Hinc II restriction site. The HBVSAg/PUC18 plasmid thus obtained was propagated in an E. coli DHSα strain. 10 μg of this plasmid, isolated by plasmid extraction from E. coli, was subsequently digested with restriction enzyme EcoRI in 20 μl of 50 mM NaCl, 100 mM Tris HCI (pH 7.5), 10 mM MgCl₂, and 0.025% Triton X-100, generating a ˜3 kb fragment that contained the HBVSAg segment. The digested product was diluted to 5×10¹⁰ copies/μl. Shown below is the sequence of one of the two strands of the DNA probe. This sequence contains a “clamp” region (shown in boldface), which provides a distinct energy barrier to keep the two strands from dissociating.

| | | |
|---|---|---|
| 1 | aattcgtaat catggtcata gctgtttcct gtgtgaaatt gt tatccgct cacaattcca | (SEQ ID NO.: 3) |
| 61 | cacaacatac gagccggaag cataaagtgt aaagcctggg gtgcctaatg agtgagctaa | |
| 121 | ctcacattaa ttgcgttgcg ctcactgccc gctttccagt cgggaaacct gt cgtgccag | |
| 181 | ctgcattaat gaatcggcca acgcgcgggg agaggcggtt tgcgtattgg gcgctcttcc | |
| 241 | gcttcctcgc tcactgactc gctgcgctcg gtcgttcggc tgcggcgagc ggtatcagct | |
| 301 | cactcaaagg cggtaatacg gttatccaca gaatcagggg ataacgcagg aaagaacatg | |
| 361 | tgagcaaaag gccagcaaaa ggccaggaac cgtaaaaagg ccgcgttgct ggcgtttttc | |
| 421 | cataggctcc gcccccctga cgagcatcac aaaaatcgac gctcaagtca gaggtggcga | |
| 481 | aacccgacag gactataaag ataccaggcg tttccccctg gaagctccct cgtgcgctct | |
| 541 | cctgttccga ccctgccgct taccggatac ctgtccgcct ttctcccttc gggaagcgtg | |
| 601 | gcgctttctc atagctcacg ctgtaggtat ctcagttcgg tgtaggtcgt tcgctccaag | |
| 661 | ctgggctgtg tgcacgaacc ccccgttcag cccgaccgct gcgccttatc cggtaactat | |
| 721 | cgtcttgagt ccaacccggt aagacacgac ttatcgccac tggcagcagc cactggtaac | |
| 781 | aggattagca gagcgaggta tgtaggcggt gctacagagt tcttgaagtg gtggcctaac | |
| 811 | tacggctaca ctagaaggac agtatttggt atctgcgctc tgctgaagcc agttaccttc | |
| 901 | ggaaaaagag ttggtagctc ttgatccggc aaacaaacca ccgctggtag cggtggtttt | |
| 961 | tttgtttgca agcagcagat tacgcgcaga aaaaaaggat ctcaagaaga tcctttgatc | |
| 1021 | ttttctacgg ggtctgacgc tcagtggaac gaaaactcac gttaagggat tttggtcatg | |
| 1081 | agattatcaa aaaggatctt cacctagatc cttttaaatt aaaaatgaag ttttaaatca | |
| 1111 | atctaaagta tatatgagta aacttggtct gacagttacc aatgcttaat cagtgaggca | |
| 1201 | cctatctcag cgatctgtct atttcgttca tccatagttg cctgactccc cgtcgtgtag | |
| 1261 | ataactacga tacgggaggg cttaccatct ggccccagtg ctgcaatgat accgcgagac | |
| 1321 | ccacgctcac cggctccaga tttatcagca ataaaccagc cagccggaag ggccgagcgc | |
| 1381 | agaagtggtc ctgcaacttt atccgcctcc atccagtcta ttaattgttg ccgggaagct | |
| 1111 | agagtaagta gttcgccagt taatagtttg cgcaacgttg ttgccattgc tacaggcatc | |
| 1501 | gtggtgtcac gctcgtcgtt tggtatggct tcattcagct ccggttccca acgatcaagg | |
| 1561 | cgagttacat gatcccccat gttgtgcaaa aaagcggtta gctccttcgg tcctccgatc | |
| 1621 | gttgtcagaa gtaagttggc cgcagtgtta tcactcatgg ttatggcagc actgcataat | |
| 1601 | tctcttactg tcatgccatc cgtaagatgc ttttctgtga ctggtgagta ctcaaccaag | |
| 1711 | tcattctgag aatagtgtat gcggcgaccg agttgctctt gcccggcgtc aatacgggat | |
| 1801 | aataccgcgc cacatagcag aactttaaaa gtgctcatca ttggaaaacg ttcttcgggg | |
| 1861 | cgaaaactct caaggatctt accgctgttg ggatccagtt cgatgtaacc cactcgtgca | |
| 1921 | cccaactgat cttcagcatc ttttactttc accagcgttt ctgggtgagc aaaaacagga | |
| 1981 | aggcaaaatg ccgcaaaaaa gggaataagg gcgacacgga aatgttgaat actcatactc | |
| 2011 | ttcctttttc aatattattg aagcatttat cagggttatt gtctcatgag cggatacata | |
| 2101 | tttgaatgta tttagaaaaa taaacaaata ggggttccgc gcacatttcc ccgaaaagtg | |
| 2161 | ccacctgacg tctaagaaac cattattatc atgacattaa cctataaaaa taggcgtatc | |
| 2221 | acgaggccct ttcgtctcgc gcgtttcggt gatgacggtg aaaacctctg acacatgcag | |
| 2281 | ctcccggaga cggtcacagc ttgtctgtaa gcggatgccg ggagcagaca agcccgtcag | |
| 2341 | ggcgcgtcag cgggtgttgg cgggtgtcgg ggctggctta actatgcggc atcagagcag | |
| 2401 | attgtactga gagtgcacca tatgcggtgt gaaataccgc acagatgcgt aaggagaaaa | |
| 2461 | taccgcatca ggcgccattc gccattcagg ctgcgcaact gttgggaagg gcgatcggtg | |
| 2521 | cgggcctctt cgctattacg ccagctggcg aaagggggat gtgctgcaag gcgattaagt | |
| 2581 | tgggtaacgc cagggttttc ccagtcacga cgttgtaaaa cgacggccag tgccaagctt | |
| 2611 | gcatgcctgc aggtctcgtg gtggacttct ctcaattttc taqggggaac acccgtgtgt | |
| 2701 | cttggccaaa attcgcagtc ccaaatctcc agtcactcac caacttgttg tcctccgatt | |
| 2761 | tgtcctggtt atcgctggat gtgtctgcgg cgttttatca tctttctctt catcctgctg | |
| 2821 | ctatgcctcg gactctagag gatccccggg taccgagctc g | |

5×10² to 5×10⁻² copies of the HBV genome were resuspended in 20 μl of 1.0 M Guanidinium SCN (GUSCN) and 50 mM potassium phosphate (pH 6.0). 15 ng of the probe was then added to each of the HBV genome solution, followed by 20 gl of aniline to form a biphasic system containing a chaotropic aqueous solvent and a hydrophobic solvent. The mixture was vortexed and incubated at 30° C. for 15 minutes to allow formation of the complex.

The complex was isolated as follows. 5 M guanidinium chloride and isopropanol were added to the above mixture. After vortexing and centrifugation at 14,000 rpm for 5 minutes, the supernatant was decanted and the pellet, which contained the complex, was washed with 75% ethanol, air-dried for 10 minutes, and resuspended in 20 μl Tris-EDTA buffer.

The complex was observed as follows. 0.2 μl of PicoGreen dsDNA Quantitation Reagent (obtained from Molecular Probe, Inc catalog # P-7581) was added to 10 μl of the resuspended nucleic acid complex. 5 μl of the labeled nucleic acid complex was then applied to a microscope slide (obtained from Kevley Technologies and identified as #CFR) and the slide was air-dried overnight. The formation of conglomerate complexes were observed by microscopy at 250× magnification under both fluorescence (FIG. 2b) and optical settings (FIG. 2a)

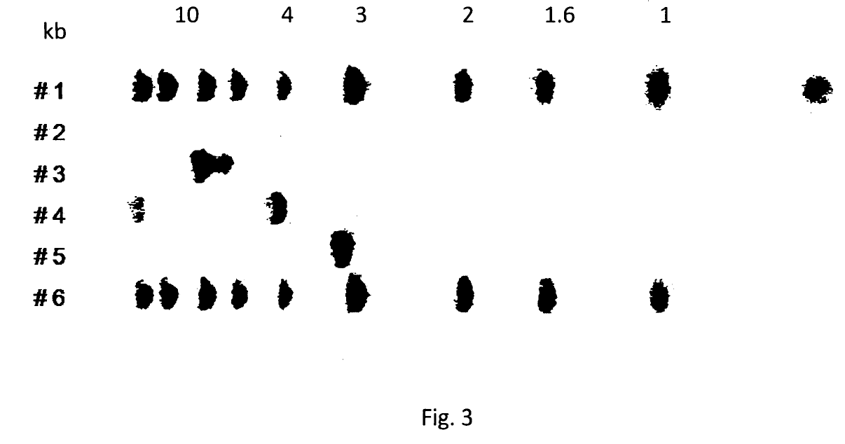

FIG. 3 demonstrates the detection of the conglomerate complex by gel electrophoresis. 10 μl of the above resuspended nucleic acid complex in TE buffer was applied to a horizontal 1% agarose gel in 0.5×TBE buffer and electrophoresed under 4 V/cm for 8 hours. The gel was then stained with 0.5 μg/ml of ethidium bromide. Photographs of the gel were taken with a red filter under UV illumination. Lane 4 shows the migration of the untreated conglomerate complex; Lane 3 shows the migration of the conglomerate complex after treated with T7 endonuclease 1; Lane 5 shows the double stranded probe HBVSAg fragment of ˜3 kb; Lane 2 shows single strand HBVSAg fragment of ˜3 kb; Lane 1 and 6 are the 1 kb sizing marker (New England Biolab Cal # N3232S). Two distinct bands were observed from Lane 4 (˜4 kb) and Lane 3 (˜10 kb) respectively. Since HBVSAg probe was ˜3 kb and the HBV genome was ˜3.2 kb, the size of the relaxed complex was higher than the combined sizes of the HBVSAg probe sequence and the HBV genome sequence.

FIG. 4 further shows that the formation of the conglomerate complex was conducted stoichiometrically. HBV genome molecules were ten fold serially diluted into 10³ to 10⁻² per reaction. Interfacial chain reactions were conducted using HBVSAg fragment as the probe, and the formed conglomerate nucleic acid complexes were collected and probe molecules were released from the complexes by digestion with T7 endonuclease I according to the manufacturer's instructions. The released HBV genome sequence is titrated by quantitative PCR. The table in FIG. 4a shows a good linear correlation between the copy number of HBV genome in the test sample and the recovered probe copy numbers in the formed conglomerate nucleic acid complex. FIG. 4b is the result of fluorescence quantitative PCR.

Further, the complex was fragmented by digesting with T7 endonuclease I as follows: 10 μl of the complex was incubated with 2 units of T7 endonuclease I (obtained from New England Biolabs, Inc. and identified as M02925) for 1 hour at 42° C. in 15 μl of 50 mM potassium acetate, 20 mM Tris acetate (pH 7.9), 10 mM magnesium acetate, 1 mM dithiothretol (OTT). The fragmented complex was used as a starting material for another round of complex formation (data not shown).

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

I claim:

1. A method for generating a conglomerate nucleic acid complex comprising of the following steps:
adsorbing a plurality of double-stranded nucleic acid probe molecules and at least one nucleic acid target molecule onto a surface, wherein the target molecules have sufficient homology to the probe molecules, and the probe molecules have a copy number that sufficiently exceeds that of the corresponding homologous target molecules; contacting the homologous target molecule with the probe molecule, whereby the target molecule triggers a chain reaction of non-reciprocal homologous recombination cross-overs among the homologous probe molecules; forming at least one conglomerate nucleic acid complex particle.

2. The method of claim 1, wherein the surface is an interfacial area between a hydrophobic and a chaotropic aqueous phase.

3. The method of claim 1, wherein the probe molecules are nucleic acids selected from a group consisting of DNA, RNA, PNA, hybrids thereof, and derivatives thereof.

4. The method of claim 3, wherein the probe molecules are paranemically denatured.

5. The method of claim 1, wherein the probe molecules are a plurality of nucleic acid molecules having homologies to a plurality of target molecules.

6. The method of claim 1, wherein the target molecules are nucleic acids selected from a group consisting of DNA, RNA and hybrids thereof.

7. The method of claim 6, wherein the target molecules are nucleic acids selected from a group consisting of single stranded, double stranded, multiple stranded nucleic acids, and a combination thereof.

8. The method of claim 7, wherein the target molecules are nucleic acids derived from a group consisting of tissue samples, body fluids, and mixtures thereof.

9. The method of claim 1, wherein the non-reciprocal homologous recombination cross-overs are assisted by protein molecules.

10. The method of claim 1, wherein the homology is reached by at least one probe molecule having contiguous homology to the primary sequences of at least one target molecules.

11. The method of claim 2, wherein adsorbing and contacting of the probe molecules with said target molecule is achieved by admixing an aqueous solution containing the target molecule with a chaotropic aqueous solution containing the probe molecules in the presence of a hydrophobic solvent.

12. The method of claim 11, wherein the hydrophobic solvent is selected from the group consisting of aniline, n-butylalcohol, tert-amylalcohol, cyclohexyl alcohol, phenol, p-methoxyphenol, benzyl alcohol, pyridine, purine, 3-aminotriazole, butyramide, hexamide, thioacetamide, 8-valarolactum, tert-butylurea, ethylenethiourea; allylthiourea, thiourea, urethane, silicones, N-propylurethane, N-methylurethane, cyanoguanidine, and a combination thereof.

13. The method of claim 1, wherein the formation of the conglomerate nucleic acid particle is detected by variations of at least one of the physical properties of at least one of the reaction components.

14. The method of claim 13, wherein the formation of the conglomerate nucleic acid particle is detected by fluorescence spectrometry.

15. The method of claim 13, wherein the formation of the conglomerate nucleic acid particle is detected by fluorescence microscopy.

16. The method of claim 1, wherein the formation reaction of the conglomerate nucleic acid complex particle is stoichiometrical between the probe molecules and the target molecules.

17. The method of claim 1, wherein the composition of the conglomerate nucleic acid particle is stoichiometrical between the probe molecules and the target molecules.

18. The method of claim 17, wherein the composition stoichiometry of the conglomerate nucleic acid particle is determined by qPCR quantification.

19. A nucleic acid complex, comprising a first pair of complementary probe strands, a second pair of the complementary probe strands, and a target sequence complementary to a site on one strand of the complementary probe strands, wherein the target sequence cross-links with one strand of the first pair via the site, and the other strand of the first-pair probe cross-links with one strand of the second-pair probe, also via the site.

20. The nucleic acid complex of claim 19, further comprising a third pair of the complementary probe strands, wherein the other strand of the second-pair probe cross-links with one strand of the third pair, also via the site.

21. The nucleic acid complex of claim 20, further comprising a plurality of pairs from the complementary probe strands, wherein each pair cross-links with another pair in the same manner as the first-pair probe cross-links with the second-pair probe.

22. The nucleic acid complex of claim 21, wherein the nucleic acid complex, when excited at 518 nm after staining with ethidium bromide, emits fluorescence at 605 nm with an intensity at least 10 times of that of non-crosslinked plurality pairs of the complementary probe strands.

23. A kit for generating a conglomerate nucleic acid complex comprising of a probe nucleic acid specific to a target sequence, a chaotropic aqueous solvent and a hydrophobic solvent.

24. A kit for detecting specific nucleic acid sequences comprising of a probe nucleic acid specific to a target sequence, a chaotropic aqueous solvent, a hydrophobic solvent, and a fluorescent dye for staining the nucleic acid sequence.
